(19) United States
(12) Patent Application Publication
(10) Pub. No.: US 2026/0060771 A1
(43) Pub. Date: Mar. 5, 2026

(54) PET HEALTH MONITORING AUXILIARY DEVICE AND METHOD

(71) Applicants: Xuan TAN, Birmingham, AL (US); Hao YU, Birmingham, AL (US); Yingyu LI, Birmingham, AL (US)

(72) Inventors: Xuan TAN, Birmingham, AL (US); Hao YU, Birmingham, AL (US); Yingyu LI, Birmingham, AL (US)

(21) Appl. No.: 19/295,536

(22) Filed: Aug. 8, 2025

Related U.S. Application Data
(60) Provisional application No. 63/688,289, filed on Aug. 28, 2024.

(51) Int. Cl. A61B 90/00; A01K 29/00; A61B 5/145

(52) U.S. Cl. CPC A61B 90/361; A61B 5/14532; A01K 29/005; A61B 2503/40; A61B 2505/07

(57) ABSTRACT

The embodiments of this specification provide a pet health monitoring auxiliary device, comprising: an imaging module, configured to capture target images during pet health monitoring; and a control module, configured to recognize detection information based on the captured target images and control a tracking of a target object by the imaging module.

### CROSS-REFERENCE TO RELATED APPLICATIONS

This application claims priority to U.S. Patent Application No. 63/688,289, filed on Aug. 28, 2024; the entire contents of which are incorporated herein by reference.

### TECHNICAL FIELD

This specification relates to the field of pet supplies, and more particularly, to a pet health monitoring auxiliary device and method.

### BACKGROUND

In modern society, the number of households raising pets is steadily increasing. However, the challenge of monitoring and managing pet health needs to be addressed. For example, the incidence of pet obesity and diabetes has been on the rise in recent years. Traditionally, pet owners have had to frequently operate glucometers and test strips to measure their pets' blood glucose levels. This process is both cumbersome and prone to errors, particularly when it is not possible to simultaneously operate other devices, such as smartphones or laptops, to record data. Moreover, manually recording data is inconvenient for long-term health management and data analysis. Although there are Bluetooth-enabled glucometers on the market that automatically record data, they are often expensive and not suitable for average pet owners. Additionally, monitoring a pet's diet and water intake is crucial for managing diabetes, as food intake directly influences insulin dosage, and water intake and frequency are closely related to the condition.

Therefore, there is a need for a pet health monitoring auxiliary device and method to assist in the detection of pet health data.

### SUMMARY OF THE INVENTION

One or more embodiments of this specification provide a pet health monitoring auxiliary device, which includes: an imaging module, configured to capture target images during pet health monitoring, and a control module, configured to recognize detection information based on the captured target images and control a tracking of a target object by the imaging module.

In some embodiments, the pet health monitoring auxiliary device further includes: a lighting module, configured to provide illumination assistance to the imaging module.

In some embodiments, the control module is further configured to: automatically switch the device mode based on the target image. The device modes include at least one of: a non-monitoring mode, a blood glucose monitoring mode, an insulin monitoring mode, a weight recording mode, and a deworming recording mode.

In some embodiments, the control module is also configured to: record the recognized detection information.

In some embodiments, the imaging module includes multiple image acquisition devices positioned at different locations, configured to acquire sub-images from multiple angles during pet health monitoring. The sub-image from each angle includes at least a portion of a monitoring region of the target object. The control module is further configured to determine the target images during pet health monitoring based on the sub-images from multiple angles.

In some embodiments, the control module is configured to: merge the sub-images using a first machine learning model based on the sub-images from multiple angles to obtain the target images.

In some cases, the input of the first machine learning model also includes weight coefficients corresponding to the sub-images from different angles.

In some embodiments, the pet health monitoring auxiliary device also includes a remote module connected to a pet hospital, configured to automatically contact the pet hospital in response to receiving a user input or triggering preset conditions.

In some embodiments, the control module is further configured to: obtain environmental perception information, including at least one of: light intensity, light type, whether backlight is present, and a distance from the imaging module to the monitoring region; determine lighting adjustment parameters based on the environmental perception information using a second machine learning model; and adjust lighting parameters of the lighting module based on the lighting adjustment parameters.

In some embodiments, the recorded recognized detection information can be accessed by multiple devices.

One or more embodiments of this specification also provide a pet health monitoring auxiliary method, which includes: obtaining the target images during pet health monitoring, and recognizing the detection information based on the obtained target images and tracking the target object.

### BRIEF DESCRIPTION OF THE DRAWINGS

This specification will further explain the invention through exemplary embodiments, which will be described in detail with reference to the accompanying drawings. These embodiments are not limiting, and in these embodiments, the same reference numbers indicate the same structures, wherein:

FIG. 1 is a schematic diagram of the application scenario of a pet health monitoring auxiliary device according to some embodiments of this specification;

FIG. 2 is a schematic diagram of a pet health monitoring auxiliary device according to some embodiments of this specification;

FIG. 3 is an exemplary flowchart illustrating an adjustment of lighting parameters of a lighting module according to some embodiments of this specification;

FIG. 4 is an exemplary flowchart of a pet health monitoring auxiliary method according to some embodiments of this specification;

FIG. 5 is a schematic diagram of the structure of a control module according to some embodiments of this specification.

### DETAILED DESCRIPTION

To more clearly illustrate the technical solutions of the embodiments described in this specification, a brief introduction to the accompanying drawings required for the description of the embodiments is provided below. It is evident that the drawings described below are merely some examples of embodiments from this specification, and for those skilled in the, the application of this specification to other similar scenarios can be achieved without creative efforts based on these drawings. Unless explicitly indicated or made clear by the context, identical reference numbers in the figures represent the same structures or operations.

It should be understood that the terms “system,” “device,” “unit,” and/or “module” used herein are methods of distinguishing different levels of components, elements, parts, sections, or assemblies. However, if other terms can serve the same purpose, those terms may be substituted accordingly.

As used in this specification and the claims, unless explicitly indicated otherwise by the context, terms like “a,” “an,” “one,” and/or “the” are not limited to the singular but may also include the plural. Generally, the terms “comprises” and “includes” merely indicate the inclusion of the steps and elements explicitly identified, and these steps and elements do not constitute an exclusive list; the method or device may also include other steps or elements.

Flowcharts are used in this specification to illustrate the operations performed by the system according to embodiments of this specification. It should be understood that the preceding or subsequent operations do not necessarily need to be performed in precise sequential order. Instead, they may be processed in reverse order or simultaneously. Additionally, other operations can be added to or removed from these processes.

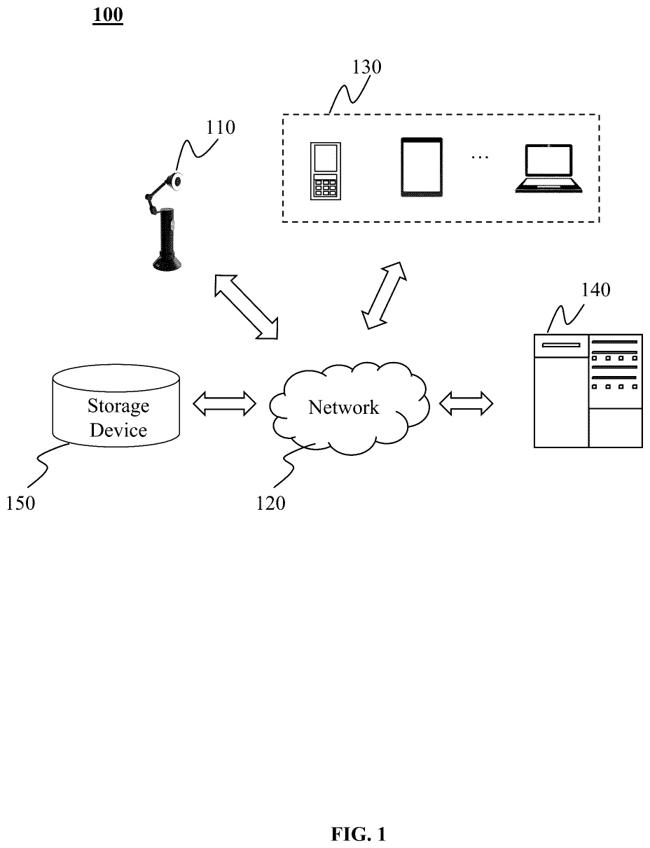

FIG. 1 is a schematic diagram of the application scenario of a pet health monitoring auxiliary device according to some embodiments of this specification.

In some embodiments, as shown in FIG. 1, the application scenario 100 of the pet health monitoring auxiliary device may include a pet health monitoring auxiliary device 110, a network 120, a terminal 130, a processor 140, and storage devices 150, among others.

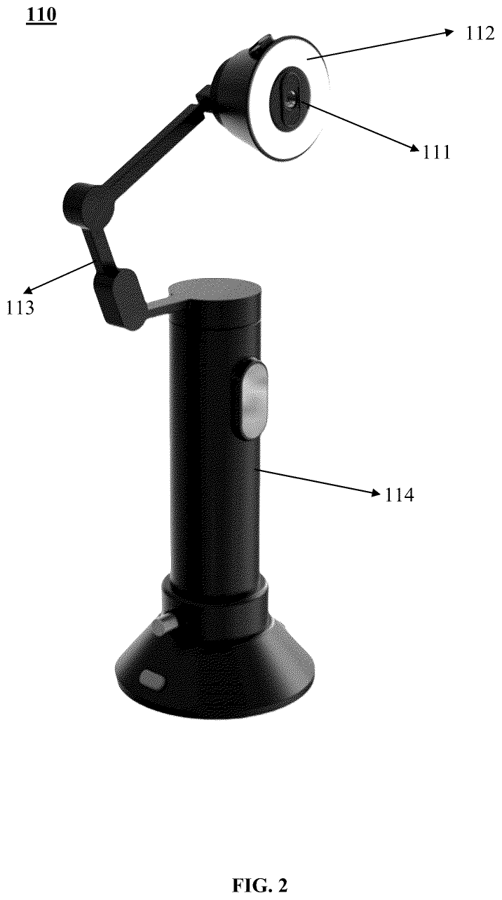

The pet health monitoring auxiliary device 110 can assist in detecting pet health-related data when a user is monitoring the pet's health. For example, it can identify and record blood glucose information when the user measures the pet's blood glucose levels. It can also measure and record the pet's daily food and water intake, among other things. More detailed explanations of the pet health monitoring auxiliary device 110 can be found in later sections, such as in FIG. 2.

The network 120 may include any suitable network that facilitates information and/or data exchange in the application scenario 100 of the pet health monitoring auxiliary device. In some embodiments, one or more components of the application scenario 100 (e.g., pet health monitoring auxiliary device 110, terminal 130, processor 140, and storage devices 150) may communicate information and/or data through the network 120 with one or more other components within the system. For example, the pet health monitoring auxiliary device 110 can send the captured target image to the processor 140 via the network 120.

The terminal 130 can provide functional components related to user interaction and can implement user interaction functions (e.g., providing or displaying information and data to the user). For instance, the user can view the pet's measurement information recorded by the pet health monitoring auxiliary device 110 through the terminal 130. The user may refer to the pet's owner, the operator of the pet health monitoring auxiliary device 110, etc. As examples, the terminal 130 may be a mobile device, tablet, laptop, desktop computer, or other devices with input and/or output capabilities, or any combination thereof.

The processor 140 can process information and/or data related to the pet health monitoring auxiliary device 110 to execute one or more functions described in this specification. In some embodiments, the processor 140 may acquire target images during pet health monitoring, recognize detection information based on the acquired target images, and track the target object. For detailed descriptions, refer to FIG. 2 and its related descriptions.

In some embodiments, the processor 140 may include a Central Processing Unit (CPU), Digital Signal Processor (DSP), System on Chip (SoC), Microcontroller Unit (MCU), computer, user console, or any combination thereof. The processor 140 may include a single server or a group of servers, which may be centralized or distributed. In some embodiments, the processor 140 may be local or remote and can be implemented on a cloud platform, which could include private, public, hybrid, community, distributed, internal, or multi-level clouds, or any combination thereof. The processor 140 may be integrated into or included as part of the pet health monitoring auxiliary device 110.

The storage device 150 can store data, instructions, and/or any other information. In some embodiments, the storage device 150 may store data obtained from the pet health monitoring auxiliary device 110 and/or processor 140, such as target images, detection information, and so on. The storage device 150 may include large-capacity storage, removable storage, volatile memory, read-only memory (ROM), or any combination thereof. It can also be implemented on a cloud platform or be part of the pet health monitoring auxiliary device 110.

It should be noted that the application scenario 100 of the pet health monitoring auxiliary device is provided for illustrative purposes only and is not intended to limit the scope of this specification. Those skilled in the art may make various changes and modifications based on the description of this specification. For example, the application scenario 100 may also include databases, information sources, etc. Additionally, the application scenario 100 can be implemented on other devices to achieve similar or different functionalities. However, these changes and modifications do not depart from the scope of this specification.

FIG. 2 is a schematic diagram of the pet health monitoring auxiliary device according to some embodiments of this specification. As shown in FIG. 2, the pet health monitoring auxiliary device 110 may include an imaging module 111, a lighting module 112, a support structure 113, and a base 114. In some embodiments, the device 110 may also include a control module (not shown in the figure). The control module may be located inside the base 114.

The imaging module 111 is configured to capture target images during pet health monitoring. In some embodiments, the imaging module 111 may include image acquisition devices such as high-definition cameras. The module can capture target images through these devices.

Pet health monitoring refers to the detection of data related to the health status of pets, such as blood glucose monitoring and insulin injection monitoring. In some embodiments, it may also include monitoring the pet's daily food and water intake.

Target images refer to the images captured during the monitoring of pet health data. The target images may vary depending on the type of health data being monitored. For example, a target image during insulin injection might include the area of the pet where the injection is administered, while for blood glucose monitoring, it might include the image of the glucometer or test strip. In some embodiments, target images can also include images captured during the pet's daily eating and drinking activities and may be in the form of photos, videos, or a combination of both.

After acquiring the target image, the imaging module 111 can send it to the control module for processing.

The control module orchestrates the operation of the various modules/structures within the pet health monitoring auxiliary device 110. In some embodiments, the control module may include a CPU, DSP, SoC, MCU, or any combination thereof.

In some embodiments, the control module is configured to recognize detection information based on the acquired target images.

Detection Information refers to the information obtained during pet health monitoring. The detection information may vary depending on the type of health data being monitored. For example, when monitoring a pet's blood glucose, the detection information may include the model of the glucometer and test strips, the pet's blood glucose levels, etc. Similarly, when administering insulin to the pet, the detection information may include the dosage and type of insulin. Additionally, when monitoring a pet's daily diet, the detection information may include the time, quantity, and frequency of the pet's drinking and eating.

In some embodiments, the control module may recognize the detection information through the target image. For instance, the control module can process the target image using a predefined image recognition method to identify the detection information.

In certain embodiments, the control module may also control the imaging module 111 to track the target object. For example, the control module can identify the target object and control the imaging module 111 to continuously track the target object in order to capture images that include the target object. The target object refers to the pet undergoing health data monitoring.

In some embodiments, the control module can also record the recognized detection information. For example, the control module can record and save the detected information in the storage device 150.

In some embodiments, the control module may also sync the recorded and saved detection information to a mobile application or cloud database.

In some embodiments, the detection information recorded by the control module can be accessed by multiple devices. For instance, the user can access and query the recorded detection information through multiple terminals via the mobile application or cloud database.

In certain embodiments, when accessing and querying the recorded detection information, the user may also correct the detection information to further improve its accuracy.

In some embodiments of this specification, the control module records and saves the detection information and syncs it to a mobile application or cloud database, allowing users to conveniently manage long-term health monitoring and data analysis, ensuring data integrity, durability, and accessibility, while reducing the risk of data loss.

In some embodiments, the pet health monitoring auxiliary device 110 can also analyze health trends based on the detection information recorded during multiple pet health monitoring sessions, further guiding the pet's health management strategy, ensuring the practical utility of the data, and supporting comprehensive pet health management.

In some embodiments, the imaging module 111 can be supported by the support structure 113 and connected to the base 114.

In certain embodiments, the support structure 113 may be immovable. It should be noted that when the imaging module 111 is capturing the target image (e.g., capturing the image during insulin injection), the pet may struggle due to pain, or there may be obstacles obstructing the view (e.g., the user's hand during insulin injection). An immovable support structure 113 restricts the angle and range of the imaging module 111, resulting in incomplete or low-resolution images.

In some embodiments, the support structure 113 may be movable. For example, the support structure 113 could be a gimbal. A movable support structure 113 allows the imaging module 111 to move freely in multiple directions, enabling stable video recording and a broader range of angles to capture more complete and clear target images.

In some embodiments, the control module can control the movement of the imaging module 111 based on the support structure 113 to capture unobstructed target images. For instance, when the control module detects that the imaging module's captured image has an obstructed monitoring region, it can control the imaging module 111 via the support structure 113 to move until the monitoring region is no longer obstructed (or until the obstructed area is below a threshold).

In some cases, even with multiple movements of the imaging module 111 via the support structure 113, it may still be impossible to capture a complete and clear target image due to excessive obstruction of the monitoring region.

To obtain a clear and complete target image, the imaging module 111 may include multiple image acquisition devices positioned at different locations; these devices are configured to capture sub-images from multiple angles during pet health monitoring. Each sub-image from a different angle includes at least part of the monitoring region. The control module is further configured to determine the target image during pet health monitoring based on the sub-images from multiple angles.

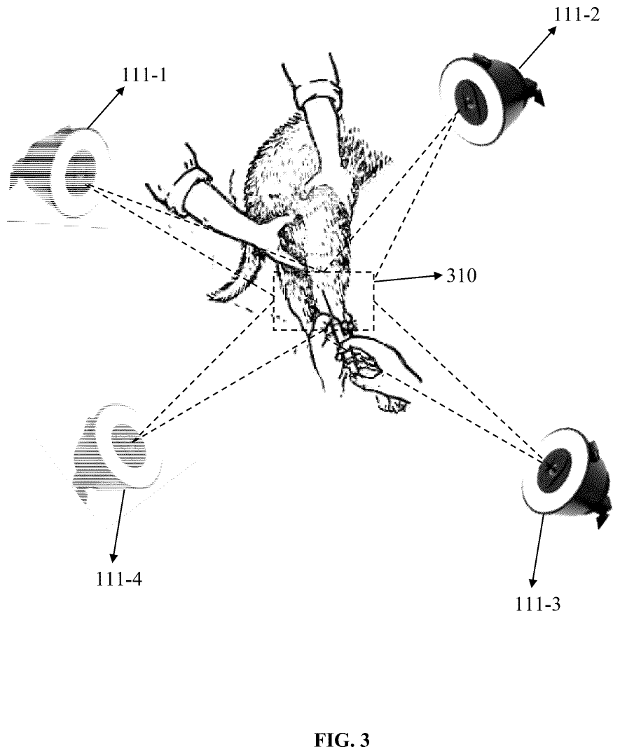

FIG. 3 is a schematic diagram showing the acquisition of the target image according to some embodiments of this specification. As shown in FIG. 3, when multiple image acquisition devices (e.g., image acquisition devices 111-1, 111-2, 111-3, 111-4, etc.) positioned at different locations individually capture target images, the monitoring region 310 may be obstructed, leading to incomplete target images and reduced accuracy in subsequent detection information recognition.

In some embodiments, the control module can control the multiple image acquisition devices to capture sub-images from multiple angles during pet health monitoring. These devices can communicate with the control module via wired or wireless methods. The control module may acquire the sub-images captured from different angles and determine the target image based on these sub-images.

In some embodiments, the control module may merge the sub-images using a first machine learning model based on the multiple angles to obtain the target image. For example, the control module can input the sub-images from different angles into the first machine learning model, which merges them to output the target image.

The input to the first machine learning model may also include weight coefficients corresponding to the sub-images from different angles.

The weight coefficient is a value that evaluates the importance of each sub-image in determining the target image. A higher weight coefficient indicates that the sub-image is more important in determining the target image.

In some embodiments, the size of the weight coefficient is related to the proportion of the monitoring region 310 in the sub-image. For example, the larger the proportion of the monitoring region 310, the greater the weight coefficient of the corresponding sub-image.

In some embodiments, the first machine learning model can be trained using historical data. The training data includes a large number of historical sub-images and their corresponding historical weight coefficients, and the training labels include the historical target images corresponding to the historical sub-images. The first machine learning model includes a neural network model, a deep neural network model, a convolutional neural network model, or the like.

In some embodiments, when a monitoring region is occluded in an image acquired by the imaging module, the control module may further perform a prediction on the image of the occluded region. In some embodiments, the control module may perform the prediction on the image of the occluded region based on an image prediction model.

The image prediction model is a machine learning model. For example, the image prediction model may be one of a neural network model, a deep neural network model, a Transformer, a 3D convolutional neural network (3D CNN) encoder, or a combination thereof.

In some embodiments, an input of the image prediction model includes: a sequence of the sub-images from multiple angles at a current time and a preset count of frames of sub-images from the same angles before the current time. For example, at the current time, the corresponding sub-images are Tt, the input of the image prediction model is an image sequence composed of a plurality of frames including Tt, Tt−1, Tt−2, etc. Tt−1, Tt−2, etc. are respectively sub-images corresponding to 1 frame before the current time, 2 frames before the current time, etc. An output of the image prediction model includes the target image and an occlusion heatmap. The target image includes an image of a predicted occluded region. The occlusion heatmap is an image that annotates the image of the predicted occluded region in the target image. For example, the occlusion heatmap may annotate (through heat values, confidence levels, etc.) reliable images of the unoccluded regions and the predicted images of the occluded regions in the target image output by the image prediction model, wherein the reliable images and the predicted images are obtained by merging the plurality of sub-images. Regions having high heat values or high confidence levels (greater than a preset heat threshold) indicate that such regions are reliable regions obtained by merging the plurality of sub-images.

In some embodiments, the image prediction model may be obtained through training. Training samples of the image prediction model include a plurality of historical image sequences (each historical image sequence being composed of a plurality of consecutive frames of historical sub-images), and training labels include historical target images and historical occlusion heatmaps.

In some embodiments of the present disclosure, the image prediction model is configured to predict images of occluded regions, and multi-view and multi-frame spatiotemporal information is fused for occlusion prediction. By introducing sequences of consecutive frames of sub-images and utilizing information before occlusion occurs or during the movement of occluding objects, the processing capability of occlusions (e.g., temporary occlusion by a user's finger) is significantly improved, and the obtained target images are more accurate. By means of the occlusion heatmap, a reliable image and a predicted image of the target image are indicated, facilitating user verification of the predicted image and subsequent repair of the target image. The occlusion heatmap may further reflect occlusion severity and information deficiency. When occlusion is severe (such as when a predicted image occupies a proportion exceeding a threshold in the target image), the control module may adjust the imaging module to minimize an area of the occluded region.

In some embodiments, the control module may further perform verification and repair on the target image output by the image prediction model to improve accuracy and reliability of the target image.

In some embodiments, the control module may construct personalized physiological characteristic maps for respective pets and store them in a cloud or a storage device (e.g., the storage device 150).

The physiological characteristic map refers to a digital structural model reflecting key physiological characteristics of a biological individual. In some embodiments, the physiological characteristic map includes vascular distribution patterns (e.g., color, thickness, orientation, or the like), skin textures, fur distribution patterns, of specific detection parts of pets (e.g., pinna vessels, dorsocervical skin) and pet-specific anatomical landmarks (e.g., specific spots, scars, bony protrusions, or the like). The physiological characteristic maps are constructed by learning from a plurality of historical detection images without occlusion or with mild occlusion (especially images of blood glucose monitoring parts and insulin injection parts). The physiological characteristic map is constructed personally for respective pets. Therefore, based on the physiological characteristic map, the image of an occluded region may be predicted and repaired according to physiological characteristics of different pets.

In some embodiments, the control module may repair the target image based on the physiological characteristic map using a repair model.

The repair model is a machine learning model. For example, the repair model may be a Conditional Generative Adversarial Network (CGAN), a diffusion model, or the like.

In some embodiments, inputs of the repair model include: boundary information of a predicted image in the target image, a region where a reliable image is located in the target image, physiological characteristic maps of the pet, and a current device mode (e.g., a blood glucose monitoring mode, an insulin monitoring mode, or the like). An output of the repair model includes a high-fidelity repaired image. The boundary information of the predicted image in the target image and the region where the reliable image is located in the target image may be determined based on the occlusion heatmap output by the image prediction model. The high-fidelity repaired image is a target image that is highly credible and closely approximates an authentic image of the pet.

In some embodiments, training samples of the repair model include: boundary information of predicted images in historical target images, regions where historical reliable images are located in the historical target images, historical physiological characteristic maps of pets, and historical device modes. The training labels include historical target images.

In some embodiments of the present disclosure, repairing the target image by utilizing personalized key physiological characteristic maps of the pet as a prior knowledge base enables the repaired target image to more closely approximate an authentic condition of the pet, thereby achieving an improved health data management effect. Furthermore, during the repair of the target image, using a current device mode as a guidance condition for repair causes the model to focus more on key information required in that mode (e.g., in a blood glucose monitoring mode, the model focuses more on blood vessels), thereby further improving accuracy of the target image.

In some embodiments, the pet health monitoring auxiliary device 110 may also include at least one of the following modes: non-monitoring mode, blood glucose monitoring mode, insulin recording mode, weight recording mode, and deworming recording mode.

The non-monitoring mode is when the pet's health data is not being monitored. In non-monitoring mode, the imaging module 111 may be shielded by a cover or similar means to protect the user's privacy. The lighting module 112 can function normally, and the pet health monitoring auxiliary device 110 can be used as a regular lighting device. The blood glucose monitoring mode, insulin recording mode, weight recording mode, and deworming recording mode correspond to the modes for monitoring health-related data such as the pet's blood glucose, injected insulin, weight, and deworming. In some embodiments, the device 110 may also include a daily monitoring mode, where it monitors the pet's daily drinking and eating data.

In some embodiments, the user can switch the mode of the pet health monitoring auxiliary device 110 via gestures. For example, the imaging module 111 can capture images including the user's gestures and send them to the control module. The control module recognizes the gesture based on the image and switches the mode accordingly. The mode of the pet health monitoring auxiliary device 110 is preset to correspond with specific gestures. For instance, a “V” gesture might activate the blood glucose monitoring mode, while an “OK” gesture might activate the insulin recording mode. In some embodiments, these gestures can be combined with regular pet training for personalized and targeted settings. For example, a pet prone to blood glucose issues might be trained to lie down with a “V” gesture, which would also activate the blood glucose monitoring mode when using the device 110, achieving a dual effect.

In some embodiments, the user can switch modes through gestures, offering a simple and direct way of operation without the need to use a touchscreen or voice control, thus avoiding startling the pet during health data monitoring. This enhances the convenience of operation and the device's animal-friendliness in practical use.

In some embodiments, the control module can automatically switch the device mode based on the target image. For example, if no image is acquired, the control module can switch to non-monitoring mode. If the target image includes key elements such as a glucometer (and/or test strips), insulin, scale, or deworming agent, the control module can switch to the corresponding blood glucose monitoring mode, insulin recording mode, weight recording mode, or deworming recording mode.

In some embodiments of this specification, automatically switching the device mode based on the target image further enhances the intelligence of the pet health monitoring auxiliary device, simplifying the operation process and making it easier and more convenient to use.

In some embodiments, the control module may further avoid anomalies caused by sudden movement of pets through an error compensation mechanism. For example, the error compensation mechanism includes image stabilization processing during sudden pet movement, and automatic lens focusing after recognizing pet movement, etc. As another example, the error compensation mechanism includes predicting the next action of the pet using the acquired images and issuing a reminder or performing error compensation on the acquisition of the images when abnormal situations occur (such as sudden movement of the pet possibly causing abnormal detection results, or the like).

In some embodiments, the control module may perform active focusing and tracking prediction for pets based on an action prediction model.

The action prediction model is a machine learning model. For example, the action prediction model is a neural network model, a deep neural network model, a long short-term memory (LSTM) network, a transformer model, or the like.

Inputs of the action prediction model include: a sequence composed of sub-images from multiple angles at a current time and a preset count of frames of sub-images from the same angles before the current time, and motion trajectories of key points of the pet (e.g., ear tips, nose tips, joints, which may be obtained via a lightweight pose estimation model) extracted in real time from the sub-images. In some embodiments, the input of the action prediction model further includes motion trends of the pet detected by an inertial measurement unit.

Outputs of the action prediction model include: predicted positions of key points of the pet in a next frame or several future frames, and a probability distribution of action states of the pet (such as “static,” “minor adjustment,” “intense struggle,” “displacement”).

The action prediction model may be obtained through training. Training samples of the action prediction model include a plurality of historical image sequences, motion trajectories of the plurality of historical key points of the pet, and historical motion trends of the pet. Training labels include historical actual positions of the pet and historical action states of the pet.

In some embodiments, the control module may pre-adjust a focusing module of the imaging module based on predicted positions of key points of the pet, such that the predicted positions of the key points are in an in-focus state when a next frame arrives, thereby significantly shortening focus lag time.

In some embodiments, the control module may further pre-adjust a direction of a gimbal based on predicted positions of key points of the pet and the probability distribution of predicted action states of the pet, thereby optimizing a field of view of the imaging module. For example, if a predicted probability of “intense struggle” of the pet is high, a field of view is pre-expanded or a wide-angle lens is switched; if a predicted probability of “static” of the pet is high, an accurate tracking is performed.

In some embodiments of the present disclosure, by using a deep learning prediction model based on time series (images, key points, IMU), not only a position of the pet is predicted but also an action state of the pet is predicted, and predictive focusing and tracking strategy adjustment are achieved by utilizing the prediction results, thereby significantly increasing an imaging success rate during high-speed movement and obtaining an improved imaging effect.

In some embodiments, the control module may further determine credibility of the recognized detection information based on a probability distribution of predicted action states of the pet and predicted positions of key points of the pet.

In some embodiments, the control module may determine credibility of the recognized detection information based on a scoring model.

The scoring model is a machine learning model. For example, the scoring model may be a lightweight classifier (e.g., a support vector machine (SVM), a decision tree) or a regression model.

In some embodiments, inputs of the scoring model may include: a probability distribution of predicted action states of the pet output by the action prediction model, predicted positions of key points of the pet, and detection information recognized in a current frame (or a current detection process); an output of the scoring model may include credibility of the detection information (e.g., a score in a range of 1-10).

The scoring model may be obtained through training. Training samples of the scoring model include probability distributions of historically predicted action states of the pet, historically predicted positions of the key points of the pet, and historically recognized detection information; the training labels include the credibility of the historical detection information.

In some embodiments, when recording detection information, the control module simultaneously stores a corresponding credibility score or an anomaly flag (e.g., reliable or unreliable). In some embodiments, during data presentation (e.g., in an app or a cloud report), for data points with low credibility or flagged as anomalies, a highlighted display is performed (such as marking in red, or annotating with a warning icon), to alert a user that the detection information may be distorted due to intense movement of the pet, and recommend the user to review or re-measure.

In some embodiments, during subsequent data analysis (e.g., a blood glucose trend graph), the control module may automatically filter out or apply weight coefficients to low-credibility detection information.

In some embodiments of the present disclosure, results of the pet behavior prediction (including predicted positions of key points of the pet and a probability distribution of predicted action states of the pet) are configured to evaluate the reliability of the detection information, perform automated flagging and subsequent processing, thereby providing essential contextual information for subsequent data interpretation and health management decisions, and significantly enhancing practicality and reliability of the data.

The lighting module 112 is configured to provide illumination assistance to the imaging module 111. In some embodiments, the lighting module 112 may include a lighting system and a projection aperture system.

The lighting system can provide illumination support for pet health monitoring. For example, during blood glucose testing or insulin injections, the lighting system can help users clearly see the pet's blood collection and injection sites.

The projection aperture system can project an aperture at the focal point of the imaging module 111 to indicate the focus location. In some embodiments, the projection aperture system can also achieve human-machine interaction through changes in the aperture color. For example, the aperture turning green indicates that the imaging module 111 has started capturing the target image, while the aperture turning red indicates a user error (such as overdosing insulin). The color changes mentioned for the projection aperture system are merely examples, and users can set other aperture colors as desired.

In some embodiments, the lighting parameters of the lighting module can be automatically adjusted. The control module may be configured to: Obtain environmental perception information. Determine light adjustment parameters based on the environmental perception information using a second machine learning model. Adjust the lighting parameters of the lighting module based on the light adjustment parameters.

Environmental perception information refers to environment-related data during pet health monitoring, particularly regarding lighting. In some embodiments, this information includes at least one of the following: light intensity, light type (e.g., natural light, artificial light), whether there is backlighting, and the distance from the imaging module to the monitoring region.

In some embodiments, environmental perception information can be obtained through sensors, such as optical sensors, distance sensors, etc.

The control module can input the environmental perception information into the second machine learning model, which outputs the light adjustment parameters. The second machine learning model may be a neural network model, a deep neural network model, or the like.

The second machine learning model can be trained using historical data, including large amounts of historical environmental perception information, with labels including historical light adjustment parameters.

Light adjustment parameters are a set of values or instructions used to adjust the light generated by the lighting module. For example, these parameters might include adjustment values or instructions for light intensity, color temperature, beam angle, and illumination range.

Lighting parameters refer to the numerical values of the light produced by the lighting module, such as light intensity, color temperature, beam angle, and illumination range.

In some embodiments of this specification, automatically adjusting the lighting parameters of the lighting module through the control module allows the lighting module to produce light that better meets actual needs, providing better illumination for both the user and the imaging module, thereby improving the clarity of the target image and the accuracy of the detected information.

In some embodiments, the pet health monitoring auxiliary device 110 may also include a remote module (not shown in the figure) connected to a pet hospital.

The remote module is a communication module for contacting a pet hospital. In some embodiments, the remote module is configured to automatically contact the pet hospital in response to receiving user input or triggering preset conditions.

Preset conditions can include various factors. Example preset conditions might include blood glucose levels exceeding a preset threshold, abnormal reactions after insulin injection (such as allergic reactions, local swelling, etc.), reduced pet activity or abnormal behavior, and significant increases or decreases in water intake or food consumption.

In some embodiments, when the pet health monitoring auxiliary device 110 receives a user input command to contact the pet hospital or when preset conditions are triggered, it can contact the hospital via voice call, video call, app, etc.

The pet health monitoring auxiliary device provided in some embodiments of this specification facilitates human-machine interaction through gesture control and changes in lighting color, simplifying the process of monitoring pet health data, and improving the accuracy and convenience of testing. The detected health data is stored in a cloud database, allowing users to easily manage long-term health monitoring and data analysis, ensuring data integrity, durability, and accessibility, and reducing the risk of data loss.

It should be noted that the above descriptions of the pet health monitoring auxiliary device and its modules are provided for convenience and do not limit the scope of this specification. It is understood that those skilled in the art, once understanding the principles of the system, may make various combinations of the modules or connect them to other subsystems without departing from these principles. For example, the various modules may share a common storage module, or each module may have its own storage module. Such variations are within the protection scope of this specification.

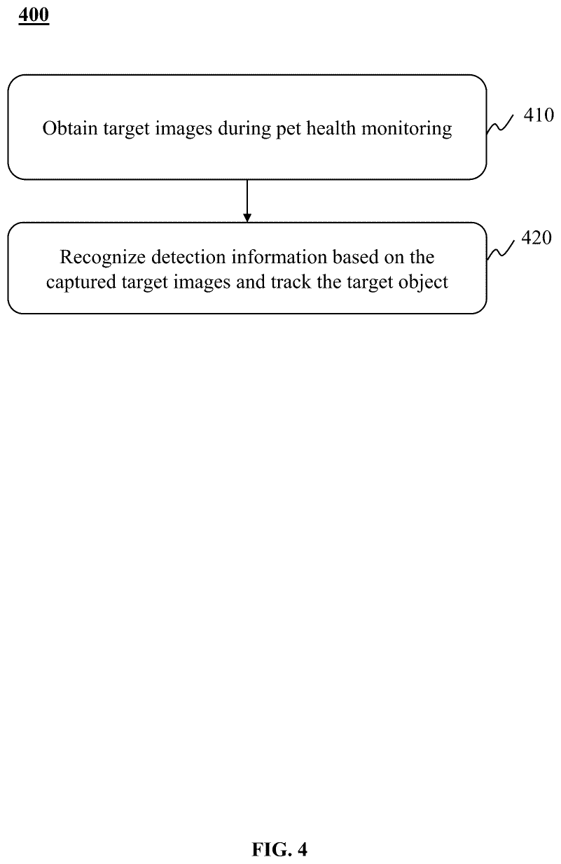

FIG. 4 is an exemplary flowchart of a pet health monitoring auxiliary method according to some embodiments of this specification. In some embodiments, the flow 400 can be executed by the processor 140 or control module. As shown in FIG. 4, the flow 400 includes the following steps:
Step 410: Obtain the target image during pet health monitoring.

The processor 140 or control module can control the imaging module 111 to capture the target image during pet health monitoring. For more details on obtaining the target image, see FIG. 2 and related descriptions.
Step 420: Recognize detection information based on the captured target image and track the target object.

The processor 140 or control module can process the target image using predefined image recognition algorithms or other methods to recognize the detection information. In some embodiments, the processor 140 or control module can also recognize the target object based on the target image and track it. The target object refers to the pet being monitored for health data. For more details, see FIG. 2 and related descriptions.

It should be noted that the description of flow 400 above is intended solely for illustration and explanation and does not limit the scope of this specification. Those skilled in the art, under the guidance of this specification, may make various modifications and changes to flow 400. However, these modifications and changes remain within the scope of this specification.

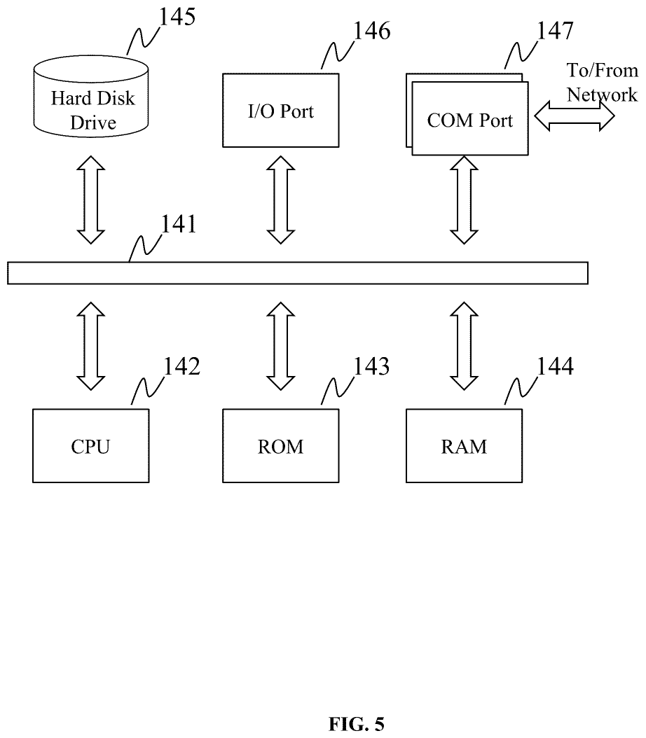

FIG. 5 is a schematic diagram of the structure of the control module according to some embodiments of this specification.

As shown in FIG. 5, the control module can include a COM port 147, which can connect to a network or be connected to it for data communication. The control module may also include a central processing unit (CPU) 142, composed of one or more processors, for executing program instructions. The computer platform may include an internal communication bus 141 and data memory (e.g., disk 145, read-only memory (ROM) 143, random-access memory (RAM) 144). The data memory is used to store various data files, different forms of programs, and program instructions that may be executed by the CPU 142. The processor 140 may also include an input/output (I/O) port 146 for input/output flow between the processor 140 and other components in the environmental data quality assessment system (such as storage device 150).

The basic concepts have been described above. It is clear that the detailed disclosure above is provided as an example and does not limit the scope of this specification. Although not explicitly stated, those skilled in the art may make various modifications, improvements, and corrections to this specification. Such modifications, improvements, and corrections are suggested within this specification, and therefore, such changes, improvements, and corrections fall within the spirit and scope of the exemplary embodiments of this specification.

Additionally, this specification uses specific terminology to describe the embodiments of this specification. The phrases “an embodiment,” “one embodiment,” and/or “some embodiments” mean that a particular feature, structure, or characteristic described in connection with the embodiment is included in at least one embodiment of this specification. Therefore, it should be emphasized and noted that “an embodiment” or “one embodiment” or “an alternative embodiment” mentioned in different places in this specification does not necessarily refer to the same embodiment. Moreover, certain features, structures, or characteristics in one or more embodiments of this specification may be appropriately combined.

Additionally, unless explicitly stated in the claims, the order of the processing elements and sequences, the use of numbers or letters, or other naming conventions in this specification are not intended to limit the order of the processes and methods of this specification. Although the disclosure above discusses some currently considered useful embodiments through various examples, it should be understood that such details are only for illustrative purposes. The appended claims are not limited to the disclosed embodiments; instead, the claims are intended to cover all modifications and equivalent combinations that fall within the spirit and scope of the embodiments of this specification. For example, although the system components described above can be implemented through hardware devices, they can also be implemented through software solutions, such as installing the described system on existing servers or mobile devices.

Likewise, it should be noted that to simplify the description of the disclosure in this specification, and to aid in understanding one or more embodiments, the previous descriptions of the embodiments of this specification sometimes group multiple features into a single embodiment, figure, or description. However, this method of disclosure does not mean that the features required by the specification exceed those mentioned in the claims. In fact, the features of the embodiments are less than all the features disclosed in the individual embodiments mentioned above.

All patents, patent applications, patent publications, and other materials, such as articles, books, manuals, publications, and documents referenced in this specification, are hereby incorporated by reference in their entirety. Except for the inconsistent or conflicting application history files in this specification, documents that limit the broadest scope of the claims in this specification (currently or added later) are also excluded. It should be noted that if there is any inconsistency or conflict between the descriptions, definitions, and/or terminology used in the attached materials and the contents of this specification, the descriptions, definitions, and/or terminology used in this specification shall prevail.

Finally, it should be understood that the embodiments described in this specification are only intended to illustrate the principles of the embodiments of this specification. Other variations may also fall within the scope of this specification. Therefore, by way of example and not limitation, alternative configurations of the embodiments of this specification may be considered consistent with the teachings of this specification. Accordingly, the embodiments of this specification are not limited to the embodiments explicitly presented and described in this specification.

What is claimed is:

1. A pet health monitoring auxiliary device, comprising:
an imaging module, configured to capture target images during pet health monitoring; and
a control module, configured to recognize detection information based on the captured target images and control a tracking of a target object by the imaging module.

2. The pet health monitoring auxiliary device according to claim 1, further comprising:
a lighting module, configured to provide illumination assistance to the imaging module.

3. The pet health monitoring auxiliary device according to claim 1, wherein the control module is further configured to:
automatically switch device modes based on the target images, wherein the device modes include at least one of: a non-monitoring mode, a blood glucose monitoring mode, an insulin recording mode, a weight recording mode, and a deworming recording mode.

4. The pet health monitoring auxiliary device according to claim 1, wherein the control module is further configured to:
record the recognized detection information.

5. The pet health monitoring auxiliary device according to claim 1, wherein the imaging module comprises multiple image acquisition devices positioned at different locations; the multiple image acquisition devices are configured to capture sub-images from multiple angles during the pet health monitoring, the sub-image from each angle including at least part of a monitoring region of the target object; the control module is further configured to:
determine the target images during the pet health monitoring based on the sub-images from multiple angles.

6. The pet health monitoring auxiliary device according to claim 5, wherein the control module is further configured to:
merge the sub-images using a first machine learning model based on the sub-images from multiple angles to obtain the target images.

7. The pet health monitoring auxiliary device according to claim 6, wherein an input of the first machine learning model further includes weight coefficients corresponding to the sub-images from different angles.

8. The pet health monitoring auxiliary device according to claim 1, further comprising a remote module connected to a pet hospital, the remote module being configured to:
automatically contact the pet hospital in response to receiving a user input or triggering preset conditions.

9. The pet health monitoring auxiliary device according to claim 2, wherein the control module is further configured to:
obtain environmental perception information including at least one of: light intensity, light type, whether there is backlighting, and a distance from the imaging module to a monitoring region of the target object;
determine light adjustment parameters based on the environmental perception information using a second machine learning model; and
adjust lighting parameters of the lighting module based on the light adjustment parameters.

10. The pet health monitoring auxiliary device according to claim 4, wherein the recorded recognized detection information supports access by multiple devices.

11. A pet health monitoring auxiliary method, comprising:
obtaining target images during pet health monitoring; and
recognizing detection information based on the target images and tracking a target object.
